(19) United States
(12) Patent Application Publication
(10) Pub. No.: US 2023/0346880 A1
(43) Pub. Date: Nov. 2, 2023

(54) TOPICAL FORMULATIONS FOR TREATMENT OF HYPERGRANULATION TISSUE

(71) Applicant: UNIVERSITY OF SOUTH FLORIDA, Tampa, FL (US)

(72) Inventors: Jake Laun, Tampa, FL (US); William West, Tampa, FL (US); Benjamin Wright, Tampa, FL (US); Jenna Marek, Tampa, FL (US); Michael Bodnar, Tampa, FL (US); Kimberly Brown Maynell, Tampa, FL (US); Loryn Taylor, Tampa, FL (US); Fernando A. Hernandez Perez, Tampa, FL (US); David J. Smith, JR., Tampa, FL (US)

(21) Appl. No.: 18/175,379

(22) Filed: Feb. 27, 2023

Related U.S. Application Data
(60) Provisional application No. 63/314,334, filed on Feb. 25, 2022.

(51) Int. Cl. A61K 38/12; A61K 31/573; A61K 9/00; A61K 9/06; A61P 17/02

(52) U.S. Cl. CPC A61K 38/12; A61K 31/573; A61K 9/0014; A61K 9/06; A61P 17/02

(57) ABSTRACT

The present disclosure describes compositions for reducing hypergranulation tissue and methods of their use. The compositions comprise bacitracin, polymyxin b, and triamcinolone.

### CROSS-REFERENCE TO RELATED APPLICATIONS

This application claims the benefit of and priority to U.S. Provisional Application No. 63/314,334 filed Feb. 25, 2022, the content of which is incorporated by reference in its entirety.

### BACKGROUND OF THE INVENTION

Hypergranulation tissue is an excessive growth of granulation tissue in a wound and is a problem that is encountered in burn care when wounds take a significant time to heal. Hypergranulation tissue often presents at burn sites themselves or at skin graft donor sites. It occurs in 10-17% of patients and may be caused by an abnormal inflammatory process or infection. Many different wound care regimens have been devised to address this problem, such as silver nitrate, hypertonic saline, and hydrocortisone cream treatments. However, these methods are often painful or expensive and can lead to worsening of the hypergranulation tissue due to local inflammation and irritation. Therefore, improved methods of treating hypergranulation tissue are needed.

### SUMMARY OF THE INVENTION

The present application provides novel compositions and methods for treating hypergranulation tissue.

In one aspect, a topical formulation comprising bacitracin; polymyxin b; and triamcinolone is provided. In some embodiments, the formulation comprises a cream comprising the triamcinolone and an ointment comprising the bacitracin and the polymyxin b; and wherein the cream and the ointment are comprised at a ratio of about 1:1. In some embodiments the triamcinolone cream comprises 0.1% triamcinolone.

In another aspect, a method for treatment of hypergranulation tissue in a subject in need thereof is provided, the method comprising topically applying to the hypergranulation tissue the topical formulation described herein. In some embodiments, the method further comprises applying a nonstick gauze dressing over the hypergranulation tissue. In some embodiments, the composition is applied once per day until the hypergranulation tissue is resolved. In some embodiments, the first application is done within 24 hours of development of the hypergranulation tissue. In some embodiments, the hypergranulation tissue is in a burn wound or a skin graft donor site. In embodiments, the subject is a human.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 illustrates reduced hypergranulation after 1 month of treatment with the compositions described herein.

FIG. 2 illustrates reduced hypergranulation after 3 days of treatment with the compositions described herein.

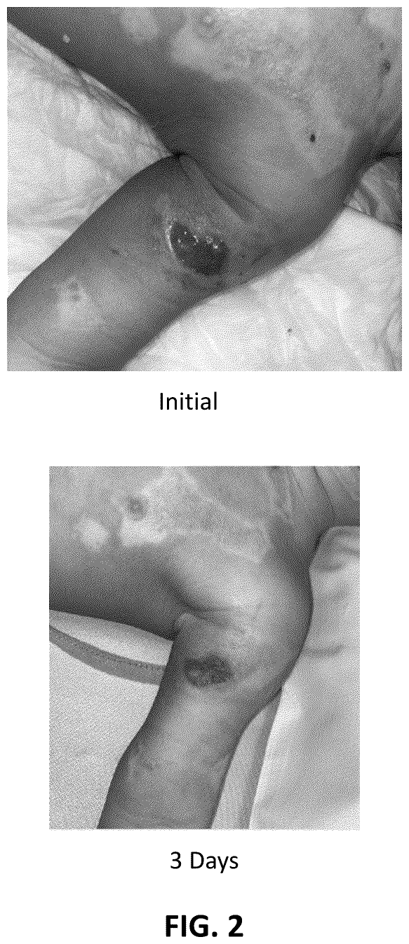

### DETAILED DESCRIPTION OF THE INVENTION

The present invention has been described in terms of one or more preferred embodiments, and it should be appreciated that many equivalents, alternatives, variations, and modifications, aside from those expressly stated, are possible and within the scope of the invention.

The inventors have found that a topical formulation comprising active ingredients bacitracin, polymyxin b, and triamcinolone reduce hypergranulation tissue. Therefore, in a first aspect of the invention, the inventors provide a composition comprising bacitracin, polymyxin b, and triamcinolone. The triamcinolone may be at a concentration of 0.1%. The compositions are formulated for topical administration. The topical formulation may comprise an ointment comprising active ingredients bacitracin and polymyxin b, and a cream comprising triamcinolone, wherein the ointment and cream are provided at a 1:1 ratio.

In a second aspect of the invention, provided herein are methods for treatment of hypergranulation tissue in a subject in need thereof, the method comprising topically applying to the hypergranulation tissue a composition comprising bacitracin; polymyxin b; and triamcinolone. The method may comprise applying the composition once per day until the hypergranulation tissue is resolved. The method may further comprise applying a nonstick gauze dressing over the hypergranulation tissue. The first application of the composition may be done within 24 hours of development of the hypergranulation tissue.

The term “subject” or “patient” are used herein interchangeably to refer to a mammal, preferably a human, to be treated by the methods and compositions described herein. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Preferably, the subject is a human. The subject may be a mammal in need of immunotherapy. The term “subject” does not denote a particular age or sex. In one specific embodiment, a subject is a mammal, preferably a human.

As used herein, “composition” or “formulation” refers to a combination of an active agent(s); and another compound or composition, inert or active, such as an adjuvant. As used herein, “pharmaceutical composition” or “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

A topical formulation refers to a combination of an active agent(s) and a vehicle. The vehicle may comprise water, oil, alcohol, propylene glycol mixed with preservatives, emulsifiers, absorption promoters, and fragrances. Topical formulations disclosed herein may comprise a lotion, gel, cream, ointment, paste, aerosol foam or spray, powder, transdermal patch, or any other substance appropriate for topical administration. The topical formulations disclosed herein may further comprise a pharmaceutically acceptable carrier or excipient.

As used herein, “pharmaceutically acceptable” can refer to compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the Food and Drug Administration.

As used herein, “pharmaceutically acceptable carrier or excipient” refers to a carrier or excipient that is useful in preparing a pharmaceutical composition that is generally safe, non-toxic and neither biologically nor otherwise undesirable, and includes a carrier or excipient that is acceptable for veterinary use as well as human pharmaceutical use. A “pharmaceutically acceptable carrier or excipient” as used herein also includes both one and more than one such carrier or excipient. Pharmaceutically acceptable carriers include, but are not limited to, diluents, preservatives, binders, lubricants, disintegrators, swelling agents, fillers, stabilizers, and combinations thereof.

As used herein, “pharmaceutically acceptable salt” can refer to any salt derived from organic and inorganic acids of a compound described herein. Pharmaceutically acceptable salt also refers to a salt of a compound described having an acidic functional group, such as a carboxylic acid functional group, and a base. Pharmaceutically acceptable salt also includes hydrates of a salt of a compound described herein.

As used herein, the terms “treat,” “treatment,” and “treating” refer to reducing the amount or severity of a particular condition, disease state, or symptoms thereof, in a subject presently experiencing or afflicted with the condition or disease state. The terms do not necessarily indicate complete treatment (e.g., total elimination of the condition, disease, or symptoms thereof). “Treatment,” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process. As used herein, the term “administering” an agent, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to a cell or subject by any suitable route for delivery of the therapeutic agent to the desired location.

As used herein, “preventative,” “preventing,” “prevent” and the like refer to partially or completely delaying or precluding the onset or recurrence of a disorder or conditions and/or one or more of its attendant symptoms or barring a subject from acquiring or reacquiring a disorder or condition or reducing a subject's risk of acquiring or reacquiring a disorder or condition or one or more of its attendant symptoms.

As used herein, “resolution” or “resolve” refer to a return from the pathological state to normal condition, e.g. 100% elimination of hypergranulation tissue.

As used herein the term “effective amount” refers to the amount or dose of a compound or composition that provides the desired effect. The effective amount may be an amount or dose of a compound or composition, upon single or multiple dose administration to the subject, that provides that treats the subject under diagnosis or treatment. An effective amount can be readily determined by those of skill in the art, including an attending diagnostician, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

As used herein, “synergistic effect,” “synergism,” or “synergy” can refer to an effect arising between two or more molecules, compounds, substances, factors, or compositions that that is greater than or different from the sum of their individual effects.

As used herein, “diluted” when used in reference to an amount of a molecule, compound, or composition including but not limited to, a chemical compound, polynucleotide, peptide, polypeptide, protein, antibody, or fragments thereof, can indicate that the sample is distinguishable from its naturally occurring counterpart in that the concentration or number of molecules per volume is less than that of its naturally occurring counterpart.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. In an embodiment, “about” indicates ±5% of the reference value. In another embodiment, “about” indicates ±10% of the reference value. In another embodiment, “about” indicates ±1% of the reference value.

As used herein, “additive effect” can refer to an effect arising between two or more molecules, compounds, substances, factors, or compositions that is equal to or the same as the sum of their individual effects.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

The invention will be more fully understood upon consideration of the following non-limiting examples.

The present invention has been described in terms of one or more preferred embodiments, and it should be appreciated that many equivalents, alternatives, variations, and modifications, aside from those expressly stated, are possible and within the scope of the invention.

The following Examples are offered for illustrative purposes only and are not intended to limit the scope of the present invention in any way. Indeed, various modifications of the invention in addition to those shown and described herein will become apparent to those skilled in the art from the foregoing description and the following examples and fall within the scope of the appended claims.

### EXAMPLES

The following Examples are illustrative and should not be interpreted to limit the scope of the claimed subject matter.

### Example 1

Methods:

96 patients admitted to the hospital for burns were identified as having developed hypergranulation tissue. A mixture of polysporin (containing bacitracin and polymyxin b) and 0.1% triamcinolone cream (0.1% triamcinolone acetonide). The products were mixed at a 1:1 ratio into a slurry and applied over the area of hypergranulation tissue and then dressed with a nonstick gauze dressing. When the patients were released from the hospital, they were instructed to apply the slurry once per day. Patients were seen every two weeks.

Results:

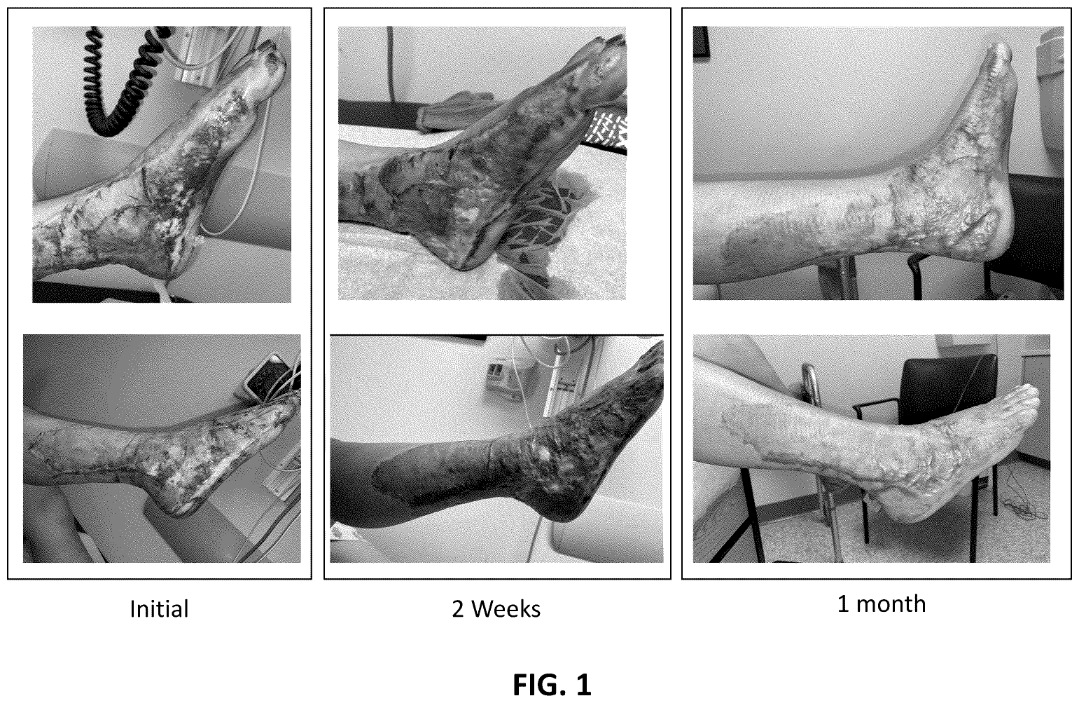

The inventors saw improvement in areas of hypergranulation tissue within 48-72 hours from initial application, and noted improvement in the extent of the wounds. Improvement was noted as clinical reduction of the hypergranulation tissue and size of the wound until epithelialized. As shown in Table 1 below, the areas of hypergranulation tissue showed complete resolution of hypergranulation tissue by an average of 31.1 days. No patients reported any significant adverse effects. FIG. 1 shows images of hypergranulation improvement in a patient using the polysporin and triamcinolone cream over the course of one month.

TABLE 1
Hypergranulation tissue improvement and recovery

| Patients | 96 | |
|---|---|---|
| Age Range | 19-89 | years old |
| Average Age | 50 | years old |
| Time to Improvement Rane | 1-112 | days |
| Average Time to Improvement | 29.9 | days |
| Time to Resolution Range | 1-149 | days |
| Average Time to Resolution | 31.1 | days |

1. A topical formulation comprising bacitracin; polymyxin b; and triamcinolone.

2. The formulation of claim 1, wherein the formulation comprises a cream comprising the triamcinolone and an ointment comprising the bacitracin and the polymyxin b; and wherein the cream and the ointment are comprised at a ratio of about 1:1.

3. The formulation of claim 2, wherein the triamcinolone cream comprises 0.1% triamcinolone.

4. A method for treatment of hypergranulation tissue in a subject in need thereof, the method comprising:
topically applying to the hypergranulation tissue the topical formulation of claim 1.

5. The method of claim 4, further comprising applying a nonstick gauze dressing over the hypergranulation tissue.

6. The method of claim 4, wherein the composition is applied once per day until the hypergranulation tissue is resolved.

7. The method of claim 4, wherein the first application is done within 24 hours of development of the hypergranulation tissue.

8. The method of claim 4, wherein the hypergranulation tissue is in a burn wound or a skin graft donor site.

9. The method of claim 4, wherein the subject is a human.
